(19) United States
(12) Patent Application Publication
(10) Pub. No.: US 2023/0165497 A1
(43) Pub. Date: Jun. 1, 2023

(54) CONTACT STIMULUS CALCULATION APPARATUS, CONTACT STIMULUS PRESENTATION APPARATUS, CONTACT STIMULUS CALCULATION METHOD, CONTACT STIMULUS PRESENTATION METHOD, AND PROGRAM

(71) Applicant: NIPPON TELEGRAPH AND TELEPHONE CORPORATION, Tokyo (JP)

(72) Inventors: Mana SASAGAWA, Musashino-shi, Tokyo (JP); Daiki SATO, Musashino-shi, Tokyo (JP); Arinobu NIIJIMA, Musashino-shi, Tokyo (JP); Tomoki WATANABE, Musashino-shi, Tokyo (JP)

(73) Assignee: NIPPON TELEGRAPH AND TELEPHONE CORPORATION, Tokyo (JP)

(21) Appl. No.: 17/923,980

(22) PCT Filed: May 15, 2020
(86) PCT No.: PCT/JP2020/019557

§ 371 Date: Nov. 8, 2022

(51) Int. Cl. A61B 5/16; A61B 5/00

(52) U.S. Cl. CPC A61B 5/165; A61B 5/0048

(57) ABSTRACT

A larger number of emotions are expressed by a contact stimulus. An input unit into which a type of an emotion and a degree of a strength of the emotion are input, and a calculation unit that calculates a presentation pattern of a contact stimulus and a degree of a strength of the contact stimulus in accordance with content input into the input unit are included.

### TECHNICAL FIELD

The present invention relates to a contact stimulus calculation device, a contact stimulus presentation device, a contact stimulus calculation method, a contact stimulus presentation method, and a program.

### BACKGROUND ART

In recent years, a technology of expressing emotions of robots has been focused on in order to smoothly perform interaction between robots and humans. In particular, attempts of expressing emotions of a robot by giving a contact stimulus to a human by changing the shape of a contact portion between the robot and the human (see Non-Patent Literature 1) or the contact pattern of the robot with respect to the human (see Non-Patent Literature 2) have been made by taking a case where the robot does not have an anthropomorphic form into consideration.

### CITATION LIST

### Non-Patent Literature

Non-Patent Literature 1: Y. Hu and G. Hoffman, “Using skin texture change to design emotion expression in social robots,” in 2019 14th ACM/IEEE International Conference on Human-Robot Interaction (HRI). IEEE, 2019, pp. 2-10.

Non-Patent Literature 2: Lawrence H. Kim and Sean Follmer. 2019. SwarmHaptics: Haptic Display with Swarm Robots. In Proceedings of the 2019 CHI Conference on Human Factors in Computing Systems (CHI '19). Association for Computing Machinery, New York, N.Y., USA, Paper 688, 1-13.

### SUMMARY OF THE INVENTION

### Technical Problem

As Russell's circumplex model indicates, there are various human emotions. However, in the current related art including the technologies described in Non-Patent Literatures described above, there is a problem in that the types of the emotions that can be expressed are limited because the contact stimuli that can be presented are limited. For example, in the technology described in Non-Patent Literature 1, there is a problem in that it is difficult to express the emotion of “sad”. There is also a problem in that it is difficult to express emotions mapped in close positions on Russell's circumplex model, for example, “sad” and “bored” while distinguishing those emotions from each other.

The present invention has been made in view of the situation as above and an object thereof is to provide a contact stimulus calculation device, a contact stimulus presentation device, a contact stimulus calculation method, a contact stimulus presentation method, and a program capable of expressing a larger number of emotions by a contact stimulus.

### Means for Solving the Problem

One aspect of the present invention includes: an input unit into which a type of an emotion and a degree of a strength of the emotion are input; and a calculation unit that calculates a presentation pattern of a contact stimulus and a degree of a strength of the contact stimulus in accordance with content input into the input unit.

### Effects of the Invention

According to one aspect of the present invention, a larger number of emotions can be expressed by the contact stimulus.

### BRIEF DESCRIPTION OF DRAWINGS

FIG. 1 is a block diagram illustrating the functional configuration of a contact stimulus presentation device according to one embodiment of the present invention.

FIG. 2 is a diagram showing specific examples of various parameters stored in an emotion DB according to the same embodiment.

FIG. 3 is a view describing the external appearances and the usage environments of robots according to the same embodiment.

FIG. 4 is a view illustrating contact materials to be installed on the robots according to the same embodiment in a removed state.

FIG. 5 is a view describing a specific example of two types of contact patterns according to the same embodiment.

FIG. 6 is a view illustrating an example realized by a specific hardware configuration according to the same embodiment.

FIG. 7 is a diagram showing experiment results of contact stimuli according to the same embodiment.

### DESCRIPTION OF EMBODIMENTS

One embodiment for a case where the present invention is applied to a contact stimulus presentation device is described below.

### Configuration

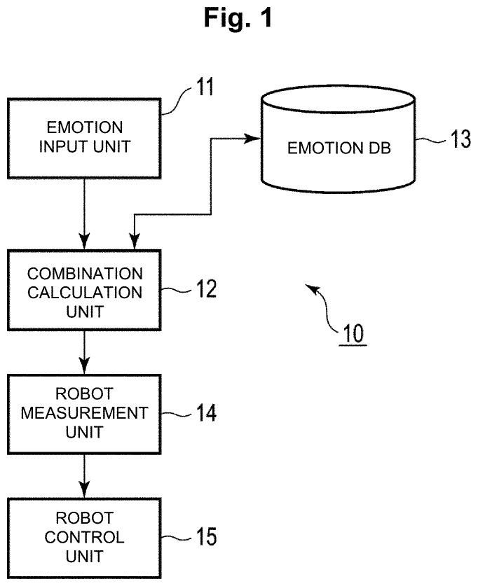

FIG. 1 is a block diagram describing the functional configuration of a contact stimulus presentation device 10 according to the present embodiment. In FIG. 1, the contact stimulus presentation device 10 includes an emotion input unit 11, a combination calculation unit 12, an emotion database (DB) 13, a robot measurement unit 14, and a robot control unit 15.

The emotion input unit 11 inputs the type of the emotion to be presented by a robot described below and the strength of the degree thereof into the combination calculation unit 12. The combination calculation unit 12 refers to the emotion DB 13 on the basis of the content input by the emotion input unit 11, reads out various parameters for presenting the contact stimulus, and outputs the various parameters to the robot measurement unit 14.

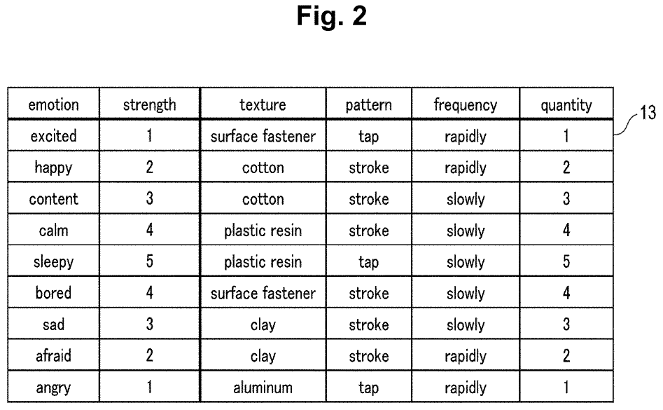

FIG. 2 is a diagram showing a specific example of a combination table of various parameters for presenting the given emotion as the contact stimulus. The various parameters are stored in the emotion DB 13 in advance.

As the combinations of the parameters, nine types, that is, “excited”, “happy”, “content”, “calm”, “sleepy”, “bored”, “sad”, “afraid”, and “angry” are defined as emotions, and five types of contact materials (textures), two types of contact patterns (patterns), two types of contact frequencies, and five stages of the degree of the strength of the contact (quantity) (the number of the robots to be used as described below) are defined in correspondence to the strengths thereof.

The nine types serving as emotions are selected such that four orthants divided by two orthogonal axes, that is, the “arousing-sleepy” axis and the “pleasure-unpleasure” axis in Russell's circumplex model are covered.

The five types of contact material properties (textures) include “synthetic resin (plastic resin)”, “aluminum”, “clay”, a “hook and loop fastener (surface fastener)”, and “toweling (cotton)”.

The two types of contact patterns (patterns) include a “point contact (tap)” and a “rotation surface contact (stroke)”.

The two types of contact frequencies (frequencies) include “slowly” of which contact frequency is low and “rapidly” of which contact frequency is high.

The robot measurement unit 14 measures the position and the facing direction of the robot serving as the target of control, and outputs the measurement result to the robot control unit 15 with various parameters received from the combination calculation unit 12.

The robot control unit 15 individually controls the operation of at least one robot serving as the target of control on the basis of the various parameters and the measurement result of the robot received from the robot measurement unit 14.

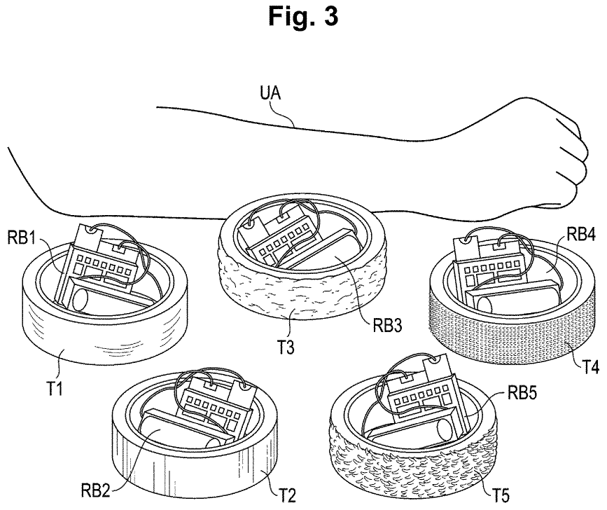

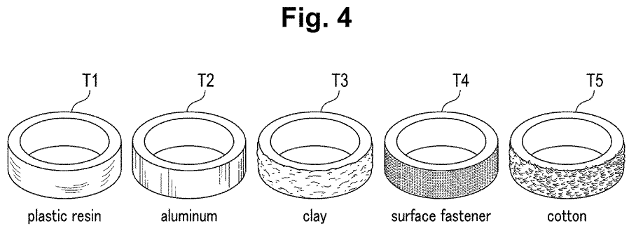

FIG. 3 and FIG. 4 are views describing external appearances of five robots RB1 to RB5 that serve as targets of control, are small in size, and self-travel on a table. All of the robots RB1 to RB5 have the same configuration except for properties of ring-shaped contact materials installed on the outer peripheral surfaces.

As the usage environment is illustrated in FIG. 3, in each of the robots RB1 to RB5, an electronic circuit, a battery serving as a power source, and the like are disposed in a casing having a bottomed cylinder shape, and the robots RB1 to RB5 are able to move and rotate on a two-dimensional surface by a motor and wheels (none shown) disposed on a bottom surface on the lower surface side thereof. The robots RB1 to RB5 each give a contact stimulus based on control by bringing, as appropriate, the contact material property installed on the outer periphery into contact with an upper arm UA of a user in accordance with the combinations shown in FIG. 2.

In FIG. 3, a state in which outer peripheral rings T1 to T5 having different contact material properties are installed on the five robots RB1 to RB5 is illustrated. However, in the present embodiment, a case where the same contact material property is installed on a plurality of robots is also supposed. Therefore, each of the outer peripheral rings T1 to T5 are prepared by a maximum number of five for the five robots RB1 to RB5.

FIG. 4 is a view illustrating the outer peripheral rings T1 to T5 of the contact material properties to be installed on the robots RB1 to RB5 in a removed state.

The outer peripheral ring T1 of the “synthetic resin (plastic resin)” is configured by an acrylic compound containing an acrylic acid monomer of which surface is smoothly processed, for example.

The outer peripheral ring T2 of “aluminum” is configured by an A1050P (pure aluminum) plate having a thickness of 0.1 [mm], for example.

The outer peripheral ring T3 of “clay” is configured by drying a lightweight resin clay mixed with water, for example.

The outer peripheral ring T4 of a “hook and loop fastener (surface fastener)” is configured by a polypropylene and polyacetal resin, for example.

The outer peripheral ring T5 of “toweling (cotton)” is configured by a cotton material of which pile is long called rabbit boa and the like of which outer surface side is raised, for example.

The outer peripheral rings T1 to T5 are members that can be installed in common on the robots RB1 to RB5, but the areas of the portions in the outer peripheral surfaces of the outer peripheral rings T1 to T5 that are actually brought into contact with the upper arm UA of the user do not necessarily need to be in common.

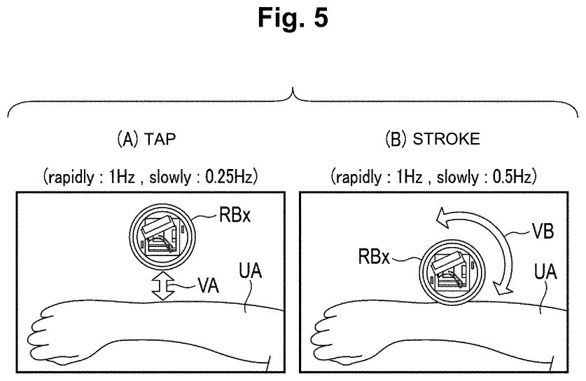

FIG. 5 is a view describing specific examples of two types of contact patterns (patterns). In the “point contact (tap)” illustrated in FIG. 5A, the outer peripheral surface is brought into point contact with the upper arm UA of the user by linearly abutting against the upper arm UA only once as indicated by arrow VA in the tap operation direction by taking the time of “0.25 [Hz] (4 [second] cycles)” in “slowly” of which contact frequency is low and the time of “1 [Hz] (1 [second] cycle)” in “rapidly” of which contact frequency is high.

In the “rotation surface contact (stroke)” illustrated in FIG. 5B, the outer peripheral surface is brought into continuous contact with the upper arm UA of the user by rotating while intentionally scratching the upper arm UA as indicated by arrow VB in the stroke operation direction by taking the time of “0.5 [Hz] (2 [second] cycles)” in “slowly” of which contact frequency is low and the time of “1 [Hz] (1 [second] cycle)” in “rapidly” of which contact frequency is high.

The specific numerical values of the contact frequencies (frequencies) described in FIG. 5 are examples of the contact patterns (patterns), and different numerical values may be settable or changeable, as appropriate, in each case depending on the contact pattern, the contact material property (texture), and the like.

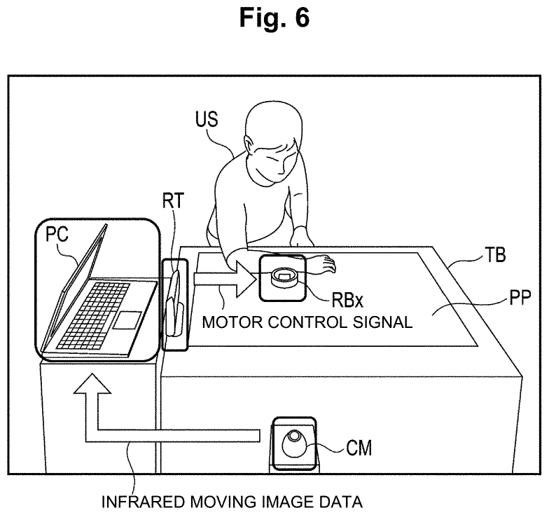

FIG. 6 illustrates an example realized by a specific hardware configuration according to the present embodiment. A robot RBx (at least one of RB1 to RB5) that comes into contact with an upper arm of a user US by moving on a table TB operates in accordance with a motor control signal transmitted from a personal computer PC via a wireless LAN router RT.

The robot RBx includes a main body casing, an electronic circuit including a microcomputer, a power source regulator, and a motor driver, a motor with gears, wheels, an infrared LED (light emitting diode), and a battery, for example. The main body casing is a casing made of synthetic resin having a bottomed cylinder shape of which outer diameter is about 80 [mm], for example, manufactured by a three-dimensional printer.

The microcomputer installed in the robot RBx has a wireless LAN function and drives the motor with gears and the wheels by transmitting a driving signal in accordance with a motor control signal transmitted from the wireless LAN router RT to the motor driver by inter-integrated circuit (I2C) communication that is synchronous serial communication. The infrared LED is disposed on the bottom surface of the main body casing on the lower surface side thereof so as to face downward and is configured to be able to measure the position and the facing direction of the robot by an infrared camera CM described below.

More specifically, three infrared LEDs are disposed on the bottom surface of the main body casing of the robot RBx. The three infrared LEDs are disposed such that the position of the center of gravity of a triangle formed by connecting the three infrared LEDs matches with the center position of the bottom surface of the main body casing and the infrared LED corresponding to the vertex of the most-acute angle of the triangle is in the front direction in terms of the control of the robot RBx.

One of the outer peripheral rings T1 to T5 of various material properties as described in FIG. 4 is selected and installed on the circumferential outer peripheral surface of the cylindrical main body casing.

The contact stimulus presentation device 10 described above is installed into the personal computer PC as an application program. As a result, the functions of the units are realized. The contact stimulus presentation device 10 transmits a motor control signal to the robot RBx via the wireless LAN router RT.

The table TB has a size in which the width is 91 [cm], the depth is 61 [cm], and the height is 67 [cm], for example. In the table TB, a surface on which the robot RBx travels is an acrylic plate and imitation Japanese vellum PP is laid thereon. The infrared camera CM is installed in a lower portion of the table TB such that the image pickup direction is upward, and constantly picks up an image of infrared light emitted from the infrared LEDs disposed on the lower surface of the robot RBx by a moving image. Infrared moving image data acquired by the infrared camera CM is transmitted to the personal computer PC.

An infrared LED included in the infrared camera CM itself and originally used for object ranging and the like is unnecessary when infrared photography is constantly performed and hence is removed. An auxiliary wide-angle lens is installed in order to assist the angle of view of the photography of the infrared camera CM, and the peripheral distortion generated in the photographed image that is acquired is removed by software separately installed in the personal computer PC.

### Operation

An operation for presenting the contact stimulus to the user is described below.

The operation as the contact stimulus presentation device 10 is realized by starting an application program installed in the personal computer PC and executing the application program on the personal computer PC.

First, as processing in the emotion input unit 11, the user US selects the type of the emotion desired to be expressed from the nine types shown in FIG. 2, selects the degree of the strength of the emotion, for example, any of the five levels from “1” to “5” as well, and then directly inputs each of the selection results by the personal computer PC.

In the personal computer PC, as the combination calculation unit 12, the combination of the material property, the contact pattern, the contact frequency, and the number of robots that is optimal for expressing the type of the emotion and the degree of the strength of the emotion that are selected is calculated.

In the present embodiment, as shown in FIG. 2, combinations of the material properties, the contact patterns, the contact frequencies, and the number of robots necessary for the type of the emotion and the degree of the strength of the emotion desired to be expressed acquired via an experiment and the like in advance are stored in advance as the emotion DB 13, and the emotion DB 13 is referred to.

A generation example of a combination table of the emotion expressions shown in FIG. 2 is described in detail.

In the beginning of the generation, the robot RBx is used as an experiment, a total of 20 types of contact stimuli, that is, “five types of material properties”33 “two types of contact patterns”×“two types of contact frequencies” are given to 13 subjects by a number of ten times each, and the subjects select and vote the emotion that the subjects feel that the robot RBx is expressing from the nine types.

Next, the combination table is generated by considering that, for each emotion, the contact stimulus with the highest number of votes by all of the subjects (the contact stimulus that does not overlap with the contact stimulus considered to be optimal for expressing another emotion when the number of votes are the same) is the optimal combination for expressing the emotion.

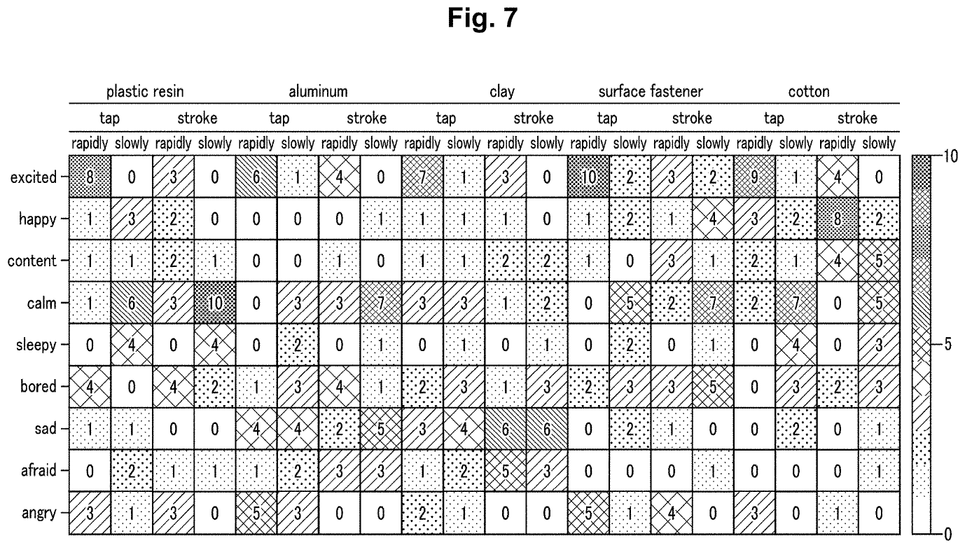

FIG. 7 shows the results of the experiment in the present embodiment. The numerical values of “0” to “10” shown in FIG. 7 are the number of votes of the subjects that have voted that the robot RBx is presenting each emotion when each contact stimulus is presented. For example, in the row of the emotion of “calm”, the contact stimulus with the highest number of votes is the combination of the contact material property of “synthetic resin (plastic resin)”×the contact pattern of “rotation surface contact (stroke)”×the contact frequency of “slowly” with “10” votes.

Therefore, in order to express the emotion of “calm”, the abovementioned combination, that is, the contact material property of “synthetic resin (plastic resin)”×the contact pattern of “rotation surface contact (stroke)”×the contact frequency of “slowly” is considered to be optimal and is incorporated into the combination table to be stored in the emotion DB 13 as shown in FIG. 2. The same applies to other emotions. The combination considered to be optimal is determined and is incorporated into the combination table to be stored in the emotion DB 13 as shown in FIG. 2.

The optimal number of the robots RBx for expressing the degree of the strength of the emotion is further added to the combination table shown in FIG. 2. In the present embodiment, the degree of the strength of the emotion is in five stages, and it is considered that the same number, that is, one to five of the robots RBx are optimal for the expression. Therefore, when “happy” is desired to be expressed, for example, the outer peripheral rings T5 of “toweling (cotton)” are installed on two of the robots RBx.

An appropriate combination in accordance with the emotion input from the emotion DB 13 is read out. Next, as processing in the robot measurement unit 14, the positions and the facing directions of the robots RB1 to RB5 at the present moment are measured. In the measurement, images of the infrared LEDs disposed on each of lower bottom surfaces of the robots RB1 to RB5 are picked up as moving images by the infrared camera CM below the robots RB1 to RB5. As a result, the personal computer PC that has received the infrared moving image data from the infrared camera CM calculates the positions and the facing directions of the robots RB1 to RB5.

On each of the lower bottom surfaces of the robots RB1 to RB5, three infrared LEDs are disposed so as to draw a triangle having vertices with acute angles as described above. Therefore, the position of the robot RBx can be measured from the position of center of gravity of the triangle, and the facing direction can be measured from the orientation of the most-acute angle. Further, it can be calculated which of the outer peripheral rings T1 to T5 having which contact material property is installed on the robot from the silhouette shape.

After the robot RBx having the outer peripheral ring of the contact material property that is calculated is recognized, as processing of the robot control unit 15, the robot RBx on which the appropriate outer peripheral ring is installed is moved to the position of the upper arm UA of the user US and is operated so as to give a contact stimulus to the upper arm UA of the user in accordance with the contact pattern, the contact frequency, and the number of the robots RBx that are calculated. In the present embodiment, the position of the upper arm UA of the user is determined on the table TB in advance, and the experiment is performed in a manner in which the user US is made to place the upper arm UA in a fixed position. However, when the position of the upper arm UA can be measured from the silhouette shape by image pickup by the infrared camera CM, the position at which the robot RBx is caused to operate can be accordingly adjusted.

In the abovementioned embodiment, a case where one robot RBx can provide one contact material property as a result of installing the outer peripheral rings T1 to T5 having different contact material properties on the plurality of robots RB1 to RB5 by a freely-selected combination is described, but the present invention is not limited thereto.

For example, when the “rotation surface contact (stroke)” of the contact pattern (pattern) is the performance of operation control of bringing the outer peripheral ring into contact while rotating the outer peripheral ring with use of a range within 180 degrees and equivalent to the central angle of the outer peripheral ring (including reversing the rotation direction as appropriate), one outer peripheral ring can be configured by two different contact material properties (textures). Similarly, when the “rotation surface contact (stroke)” is the performance of operation control of bringing the outer peripheral ring into contact while rotating the outer peripheral ring with use of a range within 120 degrees and equivalent to the central angle of the outer peripheral ring, one outer peripheral ring can be configured by three material properties (textures).

As above, the contact stimulus can be expressed with use of outer peripheral rings with more diverse material properties by configuring one outer peripheral ring by a plurality of contact material properties (textures) and controlling the orientation of the robot RBx such that the position in the ring that comes into contact with the upper arm UA of the user changes in correspondence to the input emotion.

The expression of the contact stimulus can be presented over a wider range by not only using material properties other than the five types described above as the contact material property (texture) but also including a mechanism capable of selecting, as appropriate, the temperature control for the outer peripheral ring, for example, heating by an electric heating material or heat absorption by a Peltier element.

### Effects of Embodiment

According to the present embodiment described in detail above, the robot can express a larger number of emotions by contact stimulus.

As the presentation patterns of the contact stimuli, the material property patterns of the contact stimuli and the operation patterns of the contact are combined with each other, and the degrees of the strength of the contact stimuli are maintained by the number of the contact stimuli. Therefore, emotion expression by more diverse and fine contact stimuli can be realized.

In particular, the material property patterns only need to be set, as appropriate, in view of the array of one or more materials and the areas of the materials, and more diverse expressions also become possible for the operation patterns of the contact depending on the way of contact and the contact frequency with respect to the upper arm UA of the user serving as the target of stimulation.

A case where the device of the present invention is realized by an application program installed in the personal computer PC illustrated in FIG. 6 has been described, but the program can be recorded on a recording medium or provided over a network.

Other than the above, the invention of the present application is not limited to the embodiment above and can be variously modified without departing from the gist thereof in the implementation phase. Various phases of the invention are included in the embodiment above, and various inventions may be extracted by suitable combinations in a plurality of constituent features that are disclosed. For example, even when some constituent features are erased from all of the constituent features described in the embodiment, the configurations for which those constituent features are erased may be extracted as the invention when the problem described in the column of “Means for Solving the Problem” can be solved and the effects described in the column of “Effects of the Invention” can be obtained.

### REFERENCE SIGNS LIST

10 Contact stimulus presentation device

11 Emotion input unit

12 Combination calculation unit

13 Emotion database (DB)

14 Robot measurement unit

15 Robot control unit

CM Infrared camera

RB1 to RB5, RBx Robot

PC Personal computer

PP Imitation Japanese vellum

RT Wireless LAN router

T1 to T5 Outer peripheral ring

TB Table

UA Upper arm (of user)

US User

VA Tap operation direction

VB Stroke operation direction

1. A contact stimulus calculation device, comprising:
an input unit into which a type of an emotion and a degree of a strength of the emotion are input; and
a calculation unit that calculates a presentation pattern of a contact stimulus and a degree of a strength of the contact stimulus in accordance with content input into the input unit.

2. The contact stimulus calculation device according to claim 1, wherein the presentation pattern of the contact stimulus is a material property pattern and a contact pattern of the contact stimulus, and the degree of the strength of the contact stimulus is a number of the contact stimuli.

3. The contact stimulus calculation device according to claim 2, wherein the material property pattern of the contact stimulus is an array of one or more materials and an area of each of the materials, and the contact pattern is a way of contact and a contact frequency with respect to a target of stimulation.

4. A contact stimulus presentation device, comprising:
the contact stimulus calculation device according to claim 1;
a measurement unit that measures positions and orientations of one or more robots and a target of stimulation; and
a control unit that controls the one or more robots such that the one or more robots present the presentation pattern of the contact stimulus and the degree of the strength of the contact stimulus that are calculated to the target of stimulation on basis of a measurement result of the measurement unit.

5. A contact stimulus calculation method, comprising:
an input step of inputting a type of an emotion and a degree of a strength of the emotion; and
a calculation step of calculating a presentation pattern of a contact stimulus and a degree of a strength of the contact stimulus in accordance with content input in the input step.

6. The contact stimulus calculation method according to claim 5, wherein the presentation pattern of the contact stimulus is a material property pattern and a contact pattern of the contact stimulus, and the degree of the strength of the contact stimulus is a number of the contact stimuli.

7. The contact stimulus calculation method according to claim 6, wherein the material property pattern of the contact stimulus is an array of one or more materials and an area of each of the materials, and the contact pattern is a way of contact and a contact frequency with respect to a target of stimulation.

8. A contact stimulus presentation method, comprising:
the contact stimulus calculation method according to claim 5;
a measurement step of measuring positions and orientations of one or more robots and a target of stimulation; and
a control step of controlling the one or more robots such that the one or more robots present the presentation pattern of the contact stimulus and the degree of the strength of the contact stimulus that are calculated to the target of stimulation on basis of a measurement result in the measurement step.

9. A non-transitory computer-readable medium having computer-executable instructions that, upon execution of the instructions by a processor of a computer, cause the computer to function as the contact stimulus calculation device according to claim 1.
